(19) United States
(12) Patent Application Publication
(10) Pub. No.: US 2014/0296650 A1
(43) Pub. Date: Oct. 2, 2014

(54) TELESCOPIC RETRACTOR HOLDER

(71) Applicant: AESCULAP AG, Tuttlingen (DE)

(72) Inventors: Dieter Weisshaupt, Immendingen (DE); Pedro Morales, Tuttlingen (DE); Andreas Elisch, Schramberg (DE); Peter Kleine, Neu-Isenburg (DE); Dominik Seyfried, Konigsfeld (DE); Robert Vogtherr, Tuttlingen (DE)

(21) Appl. No.: 14/354,563

(22) PCT Filed: Oct. 12, 2012
(86) PCT No.: PCT/EP2012/070277

§ 371 Date: Apr. 26, 2014

(30) Foreign Application Priority Data
Oct. 27, 2011 (DE) 10 2011 117 484.6

(51) Int. Cl. A61B 19/00; A61B 17/02

(52) U.S. Cl. CPC A61B 19/26; A61B 17/0206; A61B 2017/00991
USPC 600/229

(57) ABSTRACT

The present application discloses a telescopic retractor holder comprising a telescopic rod with an inner tube, an outer tube and a locking mechanism as well as a retractor rod. Said retractor rod has at its one end a retractor mounting, which is adapted for releasably gripping a retractor, and at its other end is fastened to the telescopic rod by means of a jointed connection. The telescopic rod can be supported on an operating table and has an actuating mechanism, by which the locking mechanism can be transferred from a first position, in which the inner tube and the outer tube of the telescopic rod are held immovably in relation to one another, into a second position, in which the inner tube and the outer tube of the telescopic rod are movable in relation to one another in the longitudinal direction of the tubes.

The present invention relates to a telescoping retractor holder, in particular a surgical lifting device with one-handed operation for raising a retractor for dissection of mammary arteries, for example.

In many cardiac surgical interventions, the sternum of the patient must be severed longitudinally and then the halves of the sternum spread apart with their adjoining ribs to allow the surgeon to work on the heart. Retractors are used to spread this surgical opening. When the surgery includes treatment of circulatory disturbances of the coronary vessels, a so-called bypass operation is performed. One of the most widespread surgical methods is to create a so-called arteria-mammaria bypass. In such bypass operations, a path is created around a segment of narrowed coronary artery. The arteria mammaria interna (IMA) used in this connection as a bypass vessel extends along the inner side of the thorax. A section of this artery is prepared by the surgeon, and the end is sutured to a coronary artery.

Normally, the left internal mannary artery (LIMA) is used, but often the right IMA is also used. To prepare sufficient operating room and sufficient visual conditions for the surgeon for these procedures, a lifting system is required which can vertically raise and support half of the severed sternum with the adjoining ribs.

### PRIOR ART

A wide variety of systems are known in the prior art. A first type of lifting system, disclosed in EP 0 931 509 B1 or U.S. Pat. No. 6,416,468 B2, for example, is based on a customary retractor with an adapter mounted thereon which raises the retractor on one side. In EP 0 931 509 B1, the retractor is raised using a screw with supporting plates and a thread swivel-mounted to the retractor. The lifting system is clamped or screwed to the retractor. According to U.S. Pat. No. 6,416,468 B2, the screw and the thread are replaced by a stop mechanism, but the fundamental manner of functioning is not different. To raise one side of the sternum, the support plate is pressed against the ribs, whereby the side of the sternum on which the lifting system is positioned is pulled outward.

Very similar is the second type of lifting system which operates with special blades on a retractor that is otherwise customary. Here, instead of via an adapter device, the principle of the lifting described above is achieved by employing a special blade which is flexibly attached to the retractor frame, and a spacing element. The distance from the pivoting side of the special blade to the retractor frame is set with the aid of the spacing element, which results in the lifting of the corresponding half of the sternum. Such systems are described in U.S. Pat. No. 5,025,779 A and DE 10 325 393 B3.

Also known are pure mammary retractors, which are used not for normal spreading of the sternum but only for the disection of the arteria mammaria. In DE 3 717 915 C2, such a retractor is described which functions without a separate lifting device. A blade disposed at a fixed angle to the toothed rod of the retractors is clamped into one half of the sternum, which is not raised, so that when the sternum is spread by means of the toothed drive, the retractor becomes positioned at an angle to the normal thoracic surface of the patient, and the other half of the sternum is pulled upward with the aid of special claw-shaped blades.

A technique which varies substantially from the three variants described above is the lifting of one half of the sternum by means of cables or tensile rods in conjunction with external means of attachment. This technique is described in U.S. Pat. No. 6,488,621 B1 and U.S. Pat. No. 6,689,053 B1. Here, only the desired half of the sternum is raised with the use of hooks positioned on a cable or tensile rod, with the tractive force being received by an element of attachment such as a frame or rod, which is fixed laterally on the operating table, for example. In all customary devices of this type, the user (the surgeon) must adjust to various drawbacks which, on the one hand, hamper the optimal progress of the procedure and, on the other hand, impede ideal results.

The most substantial drawback in this connection is the great forces which are exerted in lifting the sternum and bending the adjoining ribs, and which ordinarily must be absorbed by using support elements on the body of the patient. In the process, the affected half of the sternum becomes exposed, that is, bent outward, but the force required to do this is absorbed in the adjoining ribs. Due to the various lever arms, this results in very great forces being exerted on the patient. Accordingly, there is a very great risk of fracturing the sternum and ribs and injuring the neurons.

These disadvantages are not present with the technique of external attachment because, in this case, the force required to expose the half of the sternum is absorbed by the attachment system and not by the patient. With this technique, however, insufficient visual conditions are created because the technique involves only a vertical lifting of one half of the sternum, which does not provide a sufficient opening on the horizontal axis. The lack of horizontal forces does not allow use of normal blades here; rather, claw-like hooks are required, which act intensely locally on the sternum and can cause substantial fractures. In addition, such systems occupy space appreciably above the operating level, and therefore in many procedures present numerous obstacles for the surgical staff.

Another weak point is the fact that, in a case where two thoracic wall arteries (left and right) need to be prepared one after the other, shifting the lifting system to the other side is very laborious.

With devices adapted to a retractor, this shifting is relatively simple, but ordinarily requires an assistant to help the surgeon.

In contrast, retractors with special blades must be completely removed from the surgical opening and reinstalled, which necessitates interrupting the surgery and also creates a somewhat critical moment because, when the retractor is removed, the surgeon no longer has access to the heart.

In systems with an external attaching rod mounted on the operating table, there is no practical way to shift [the device] to the other side. A major consideration in this connection is that all regions vertically below the operating region are not sterile. A mounting on the operating table will be in the non-sterile region, and therefore cannot permissibly be undone to switch it to the other side. In the same way, procedural steps that need to be carried out in this region during the surgery, e.g. loosening of a clamp, are impermissible.

The object of the present invention is to create a retractor holder which enables exposure of a half of the sternum and creation of an adequate horizontal sternum opening. A further object of the invention is to create a retractor holder that may be manipulated with only one hand. A further object of the invention is to devise a retractor holder as described which can be shifted from one side of the operating table to the other with one hand, or which renders such shifting entirely unnecessary.

The object of the invention is achieved by a telescopic retractor holder according to claim 1. Additional advantageous refinements are the subject of the dependent claims.

According to one aspect of the invention, a telescopic retractor holder is provided which has an inner tube, an outer tube, and a locking mechanism, as well as a retractor rod which has a retractor mounting on one end, which retractor mounting is adapted for releasably gripping a retractor, and on its other end said retractor rod is fastened to the telescopic rod via a jointed connection. The telescopic rod can be supported on an operating table and has an actuating mechanism by which the locking mechanism can be transferred from a first position, in which the inner tube and outer tube of the telescopic rod are held immovably with respect to each other, into a second position, in which the inner tube and outer tube of the telescopic rod are movable with respect to each other in the longitudinal direction of the tubes.

In this embodiment, the retractor holder according to the invention is held in an essentially vertical position when used on a patient, wherein the retractor is attached to the sternum of the patient and the retractor mounting grips the retractor. In this way, the supporting force of the sternum, the support of the telescoping rod, and the lengths of the telescoping rod and the retractor rod result in a distinct position of the telescopic retractor holder. The essentially vertical position of the retractor holder is based on the essentially vertical orientation of the telescopic rod.

The length of the telescopic rod can be changed by unlocking the locking mechanism with the actuating mechanism, adjusting the length of the telescopic rod by raising or lowering the actuating mechanism, and then releasing the actuating mechanism, as a result of which the locking mechanism mutually fixes the adjusted position of the inner tube and the outer tube of the telescopic rod. In the simplest case, described here first, the telescopic rod is supported on the surface of the operating table. In order to prevent the telescoping rod from sliding away, depressions may be provided in the operating table.

According to another aspect of the invention, the telescopic rod may be flexibly supported on the operating table. This means that the telescoping rod need not be oriented exactly vertically with respect to the operating table, but its orientation may be adjusted to the further geometry and the conditions of engagement between the retractor and the sternum of the patient. Essentially, it is possible to rotate the retractor holder or the telescoping rod with respect to the operating table around a longitudinal axis of the operating table, so that the jointed connection between the telescopic rod and the retractor rod can be moved toward and away from the patient. If, however, the retractor is spread wider while it is being held by the retractor holder, when lifting occurs the sternum will automatically become oriented at an angle to the frontal plane, with the caudal region higher than the cranial region, because the caudal pairs of ribs in humans, for example, are longer and more flexible than the cranial pairs. This difference in height will vary depending on the exposure of the sternum, and will result in the retractor being rotated with respect to the horizontal. Therefore, the telescoping rod can rotate, at least to some degree, around the transverse axis of the operating table O, either by virtue of a rotatable mounting in this direction or a certain play in mounting, or by absorption of the rotation by the retractor rod which has a corresponding degree of freedom.

According to still another aspect of the invention, the retractor rod of the telescoping retractor holder is itself freely telescoping. Here, the term “freely telescoping” means that no locking mechanism is provided in the retractor rod, and therefore the retractor rod can freely vary in length. With such a telescoping retractor rod, the telescoping rod may be mounted on the operating table in an essentially without articulation, wherein, when the telescopic rod is lengthened, the retractor rod is also lengthened, such that the patient P is not raised at the sternum but only one side of the sternum is raised with the aid of the retractor and the retractor holder. In the telescopic device of the retractor rod, the abovementioned degree of freedom for rotation of the retractor with respect to the horizontal can then be provided, in that the freely telescoping retractor rod is unable to receive or transfer torsion forces.

According to yet another aspect of the invention, the actuating mechanism is arranged in the region of the jointed connection to the retractor rod. The jointed connection is essentially always in the plane that the retractor applies stress to. Therefore the actuating mechanism is also in this region and thus always in the sterile region. The surgeon may thus operate the retractor holder without having to undergo re-sterilization.

According to another aspect of the invention, the inner rod of the telescopic rod may be supported against an operating table, the outer tube of the telescopic rod is connected at one end to the retractor rod via the jointed connection, and the actuating mechanism is arranged in the region of the other end of the outer tube. In this case, the actuating mechanism is placed somewhat lower than in the above-described case, but is still in a region which may be deemed to be sterile.

According to another aspect of the invention, the retractor mounting is designed so as to grip a retractor in a positive fit. In this way, the forces and moments of the retractor can be well transmitted to the retractor holder, and vice versa.

According to still another aspect of the invention, the retractor mounting comprises an essentially U-shaped recess into which a frame of a retractor may be inserted, so that the retractor frame is seated at least partially against the inner wall of the recess on at least two sides. An elastically pre-stressed tongue has at its distal end a locking projection designed to rest against a retractor frame which is inserted [in the recess], thereby providing a positive fit with the retractor frame. In the simplest case, the U-shaped recess rests against a retractor component on three sides of said retractor component, and, to prevent the retractor component from sliding out of the retractor mounting, the locking projection rests against the fourth side of the retractor component. The engaged retractor component need not be rectangular. It is advantageous if the retractor component that is gripped is merely not rotationally symmetric. In this case, a force-mediated connection between the retractor and the retractor mounting must be created, which is readily possible but which complicates the surgeon's operation thereof during surgery.

According to still another aspect of the invention, the tongue is rotatably mounted on one side of the recess and is pre-stressed by an elastic component such that its distal end with the locking projection is pushed inward toward the recess. This is an advantageous arrangement because the surgeon can release the retractor mounting from the retractor by gripping the retractor rod with one hand and, for example, with the thumb of the same hand, pressing the tongue on the proximal end of the retractor rod to raise the undercut on the locking projection on the distal end of the tongue with the side of the retractor component facing away, in order to release the retractor from the retractor mounting. Conceivably, two opposite tongues may be provided which work in mirror-image fashion. Also conceivable is a tongue which is introduced into a retractor component, with the distal end of said tongue being pre-stressed away from the U-shaped recess [sic].

According to yet another aspect of the invention, the elastic component is a compression spring which presses against the proximal end of the tongue. In this case, the elastic component may be disposed in the region of the bottom of the U-shaped recess. This arrangement is also suitable for a retractor mounting having two tongues, wherein each tongue may be provided with a respective compression spring, or a common compression spring may be provided.

According to another aspect of the invention, the tongue has an actuating projection on its proximal end for releasing a retractor from the retractor mounting. This makes it easy for the surgeon to release the tongue, and also facilitates pressing the proximal end of the tongue. Under these circumstances, the tongue can be smaller because the recess for accommodating the proximal end of the tongue does not need to be able to accommodate the surgeon's thumb or another actuating finger.

According to still another aspect of the invention, the jointed connection that attaches the retractor rod to the telescopic rod is a hinge joint. A hinge joint is the simplest form of a joint and in the retractor holder according to the invention it is particularly advantageous if the retractor rod itself is freely telescoping and unable to transmit torsional forces.

According to yet another aspect of the invention, the jointed connection that connects the retractor rod to the telescopic rod is a ball joint. According to this aspect, the retractor rod may be torsionally rigid, because torsion forces are not transmitted through the ball joint.

According to another aspect of the invention, the actuating mechanism comprises a ring which is movable relative to the telescopic rod to change the locking mechanism from the first position to the second position. Here it is advantageous to have a ring which is longitudinally slidable on the tube on which said ring is provided in order to release the locking mechanism. The actuating mechanism may also be given various other forms, for example as an actuating button that is depressed in order to release the locking mechanism, or as a ring or other component that is rotated relative to the respective tube to release the locking mechanism, the rotation being around the axis of the tube or in another direction.

According to still another aspect of the invention, the telescopic rod has an essentially U-shaped attaching mount which allows it to be mounted from above onto an operating table rail. In the simplest case, this attaching mount is placed over an operating table rail without resulting in any type of positive fit or close contact. In this way, the telescopic rod can be rotated sufficiently in all directions with respect to the operating rail, and thereby can adjust to the force and moment conditions of the system.

According to yet another aspect of the invention, the cross section of the attaching mount is adjusted to a cross section of an operating table rail, such that in the applied state the attaching mount is seated at least partially against both side walls of the operating table rail. In doing this, the attaching mount is attached to the telescoping rod via a jointed connection. The rotation of the telescopic rod with respect to the operating table rail serves to safely prevent the transfer of excessive forces from the sternum to the retractor and then to the retractor holder and the operating table, and vice versa, thereby avoiding injury to the patient's sternum or the surrounding tissue. The most important rotation of the telescoping rod with respect to the operating table in this connection is the rotation around the longitudinal axis of the operating table. Rotations in other directions occur primarily as a result of misalignment of the patient on the operating table, of cutting through the sternum in other than a straight line, and of further spreading of the sternum. However, these rotations are of a magnitude that can be accommodated via play in the various supporting systems. In addition, the surgeon may always adjust the position of the support point of the telescoping rod along the longitudinal axis of the patient's body to minimize the aforementioned unintentional rotations.

According to another aspect of the invention, the jointed connection between the telescoping rod and the attaching mount is configured such that a rotational movement between the telescoping rod and the attaching mount is possible only within a predetermined angular range. This angular range is sufficiently wide to permit changes in length of the telescoping rod to be carried out from a maximum length to a minimum length or vice versa without reaching the angle stop. This angle stop serves rather to prevent the retractor mounting from being upset and entering the non-sterile region when the surgeon has already attached the attaching mount directly or indirectly to the operating table rail, but the retractor mounting has not yet been attached to the retractor. In this state, the retractor holder can tilt away from the patient without encountering an angle stop, thus tipping into the non-sterile area. With an angle stop, it can tilt away only to the extent of a predetermined angle, and the area of the retractor holder which the surgeon grips and actuates will remain within the sterile area. This makes the use of such a retractor holder even safer.

According to still another aspect of the invention, the telescopic retractor holder has at least one attaching mechanism which has an attaching rail and is adjusted to such rail so as to be releasably attachable to an operating table rail. In doing so, the telescoping rod has a fastening mounting that can be applied to or pushed onto the attaching rail. When the retractor holder is to be supported on or attached to an operating table rail, there is the problem that these operating table rails are not continuous over the entire length of the operating table O. One reason for this is that operating tables are segmented to facilitate different adjustments of the patient and the surgery. In order to close the gaps in the operating table rails, the telescopic retractor holder according to the invention has an attaching mechanism which itself can be attached to an operating table and which also has its own attaching rail on which the telescoping rod of the telescoping retractor holder can then be supported. The term “pushing on” here means pushing the fastening mounting onto the attaching rail in the longitudinal direction.

According to yet another aspect of the invention, the attaching rail has a cross section composed of a combination of an essentially circular surface and a second surface. The fastening mounting of the telescoping rod has a recess with an inner wall and two side walls, wherein the cross section of the recess essentially corresponds to the cross section of the attaching rail. The inner wall of the recess, along with the attaching rail, in the pushed-on state, gives rise to at least one undercut structure which is able to transfer tractive forces from the telescoping rod to the attaching rail. In addition, the side walls of the recess are spaced away from and oriented with respect to the lateral surfaces of the other surface of the cross sectional surface of the attaching rail such that rotation of the telescoping rod relative to the attaching rail is limited to a predetermined angular range. The cross section of the attaching rail consisting of a rotationally symmetric component and another non-rotationally symmetric component (or at least not rotationally symmetric around the same center as the first component) and the associated fastening mounting enable rotation of the telescoping rod with respect to the attaching rail within a certain angular range and at the same time prevent further rotation. The simplest combined form of this type is the shape of a keyhole consisting of a circular surface and an adjacent surface that is a segment of a circle, the latter being not fully rotationally symmetric.

According to another aspect of the present invention, the fastening mounting is disposed at one end of the telescoping rod. In this way, the lower tube of the telescoping rod and the fastening mounting may be formed from a single piece.

According to still another aspect of the invention, the retractor rod is attached to the inner tube of the telescoping rod, the actuating mechanism is provided on the inner tube of the telescoping rod, and the fastening mounting is provided on a side wall of the outer tube of the telescoping rod. With the fastening mounting being provided laterally on the outer tube of the telescoping rod, the length of the telescoping rod can be increased without the height of the system above the operating table being increased, which might interfere with the surgeon, for example limiting his field of view or of his freedom of movement. A greater length of the telescoping rod may be advantageous in particular when, with the aid of the telescoping retractor holder, not only the side of the sternum on which the retractor holder is located is to be raised by increasing the length of the telescoping rod, but also the other half of the sternum is to be raised by decreasing the length of the telescoping rod. In this process, in each case the retractor rests against the half of the sternum that is not raised, which is easily possible because it is substantially easier to bend ribs than to compress them. In order to obtain a sufficient length of the telescoping rod without the height of the system above the operating table being so great as to interfere with the surgeon, it is advantageous if the telescoping rod can be lengthened below the operating table rail. In order to nonetheless be able to attach the telescoping rod to the operating table rail, the fastening mounting must be attached laterally to the telescoping rod.

According to yet another aspect of the invention, the telescoping rod has at least one additional middle tube, wherein said middle tube is arranged between the inner tube and the outer tube so as to form with these a multiply-telescopic rod. In this way, the telescopic length can be increased without increasing the length of the telescoping rod in the collapsed state.

Additional advantages and features of the invention will be apparent to one skilled in the art from the accompanying drawings and the detailed description of the exemplary embodiments.

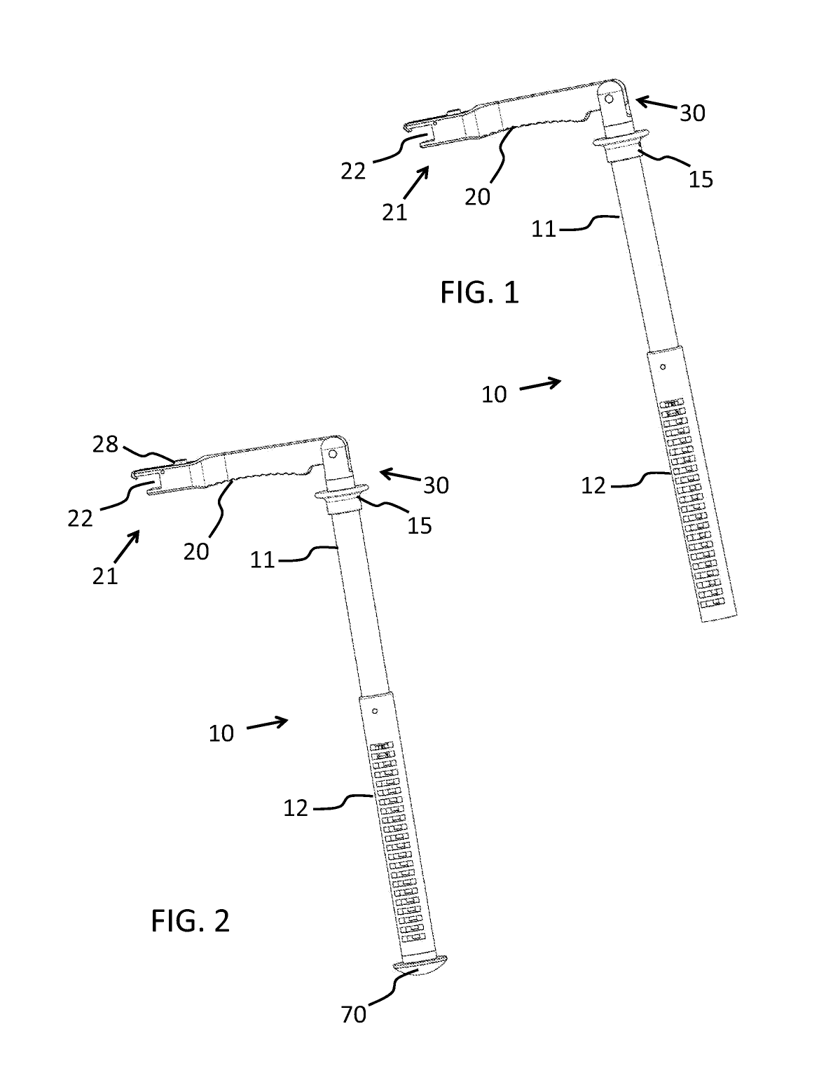

FIG. 1 illustrates a first exemplary embodiment of the telescopic retractor mounting to be supported on an operating table;

FIG. 2 illustrates a second exemplary embodiment of the telescopic retractor mounting to be supported on an operating table;

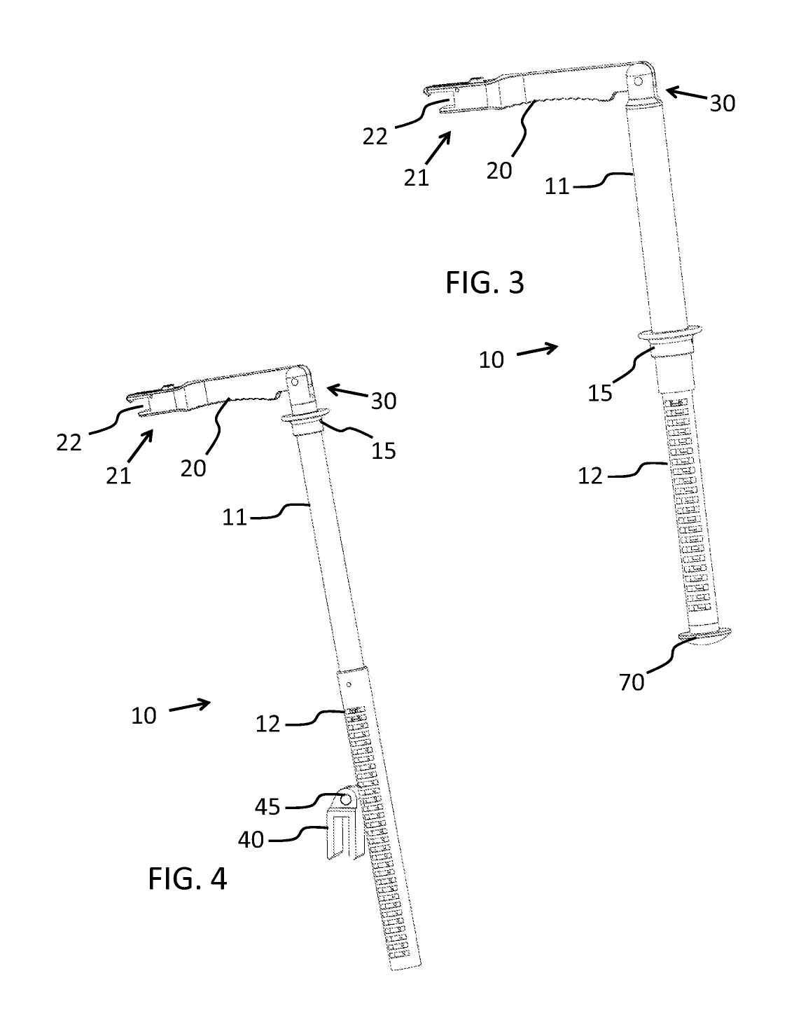

FIG. 3 illustrates a third exemplary embodiment of the telescopic retractor mounting to be supported on an operating table;

FIG. 4 illustrates a fourth exemplary embodiment of the telescopic retractor mounting to be supported on an operating table rail;

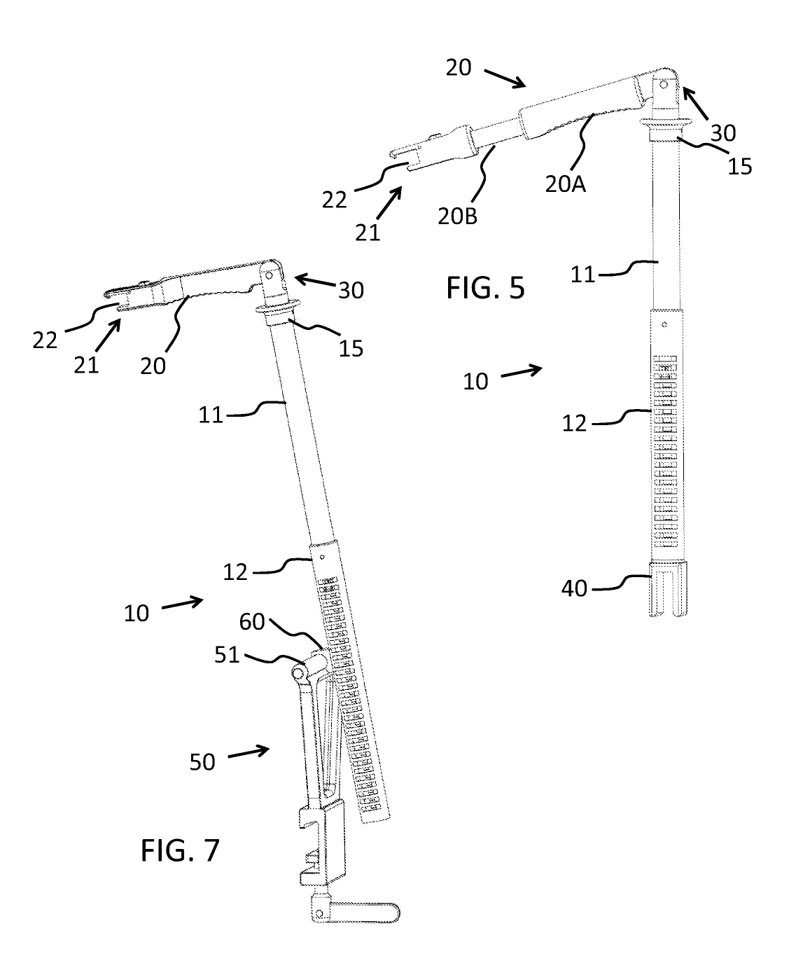

FIG. 5 illustrates a fifth exemplary embodiment of the telescopic retractor mounting to be supported on an operating table rail;

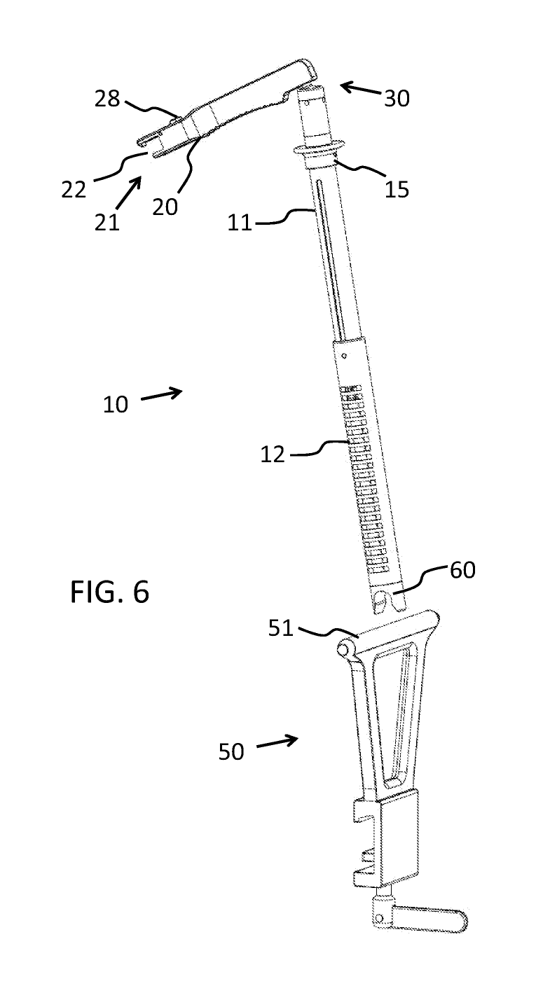

FIG. 6 illustrates a sixth exemplary embodiment of the telescopic retractor mounting with fastening mechanism;

FIG. 7 illustrates a seventh exemplary embodiment of the telescopic retractor mounting with fastening mechanism;

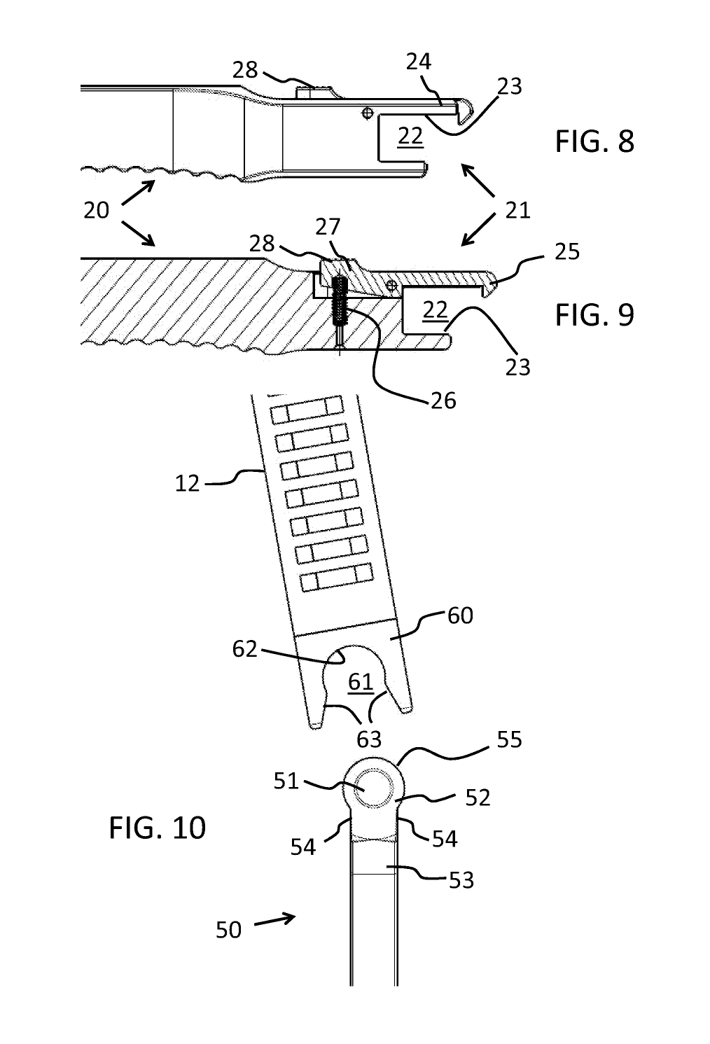

FIG. 8 is a lateral view of a retractor mounting;

FIG. 9 is a lateral cross sectional view according to FIG. 8;

FIG. 10 is a detail view of a fastening mechanism and a fastening mounting of the sixth exemplary embodiment;

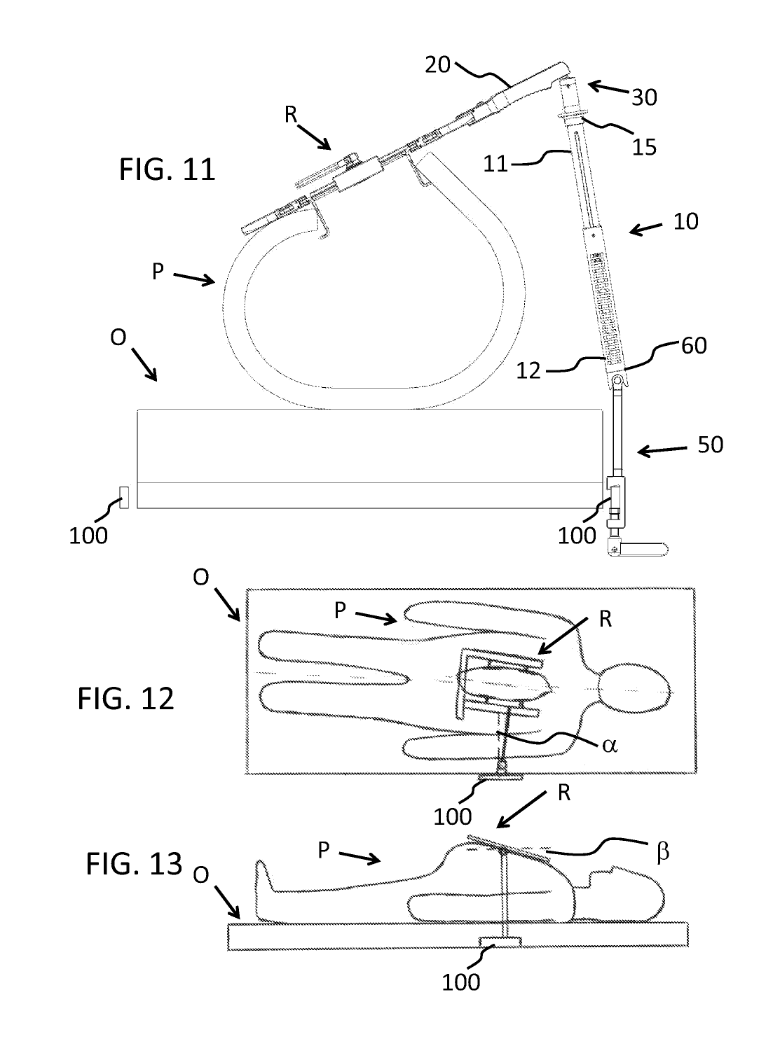

FIG. 11 is a schematic view of an operating table and the thorax of a patient, as well as a telescopic retractor mounting according to the sixth exemplary embodiment;

FIG. 12 is a view from above of a patient on an operating table; and

FIG. 13 is a lateral view of a patient on an operating table.

A first exemplary embodiment of the present invention will be described in detail with reference to FIG. 1.

The telescopic retractor holder according to the first exemplary embodiment has a telescopic rod 10 with an inner tube 11, an outer tube 12, a locking mechanism, and a retractor rod 20. The retractor rod 20 has on one end a retractor mounting 21 which can releasably grip a retractor R. On the other end, the retractor rod 20 is attached to a telescopic rod 10 via a jointed connection 30. The telescopic rod 10 according to this exemplary embodiment can be supported on an operating table O, wherein the free end of the outer tube is simply placed on the operating table O, or is inserted into special recesses in the operating table O. The telescopic rod 10 has an actuating mechanism 15 on its inner tube 11, wherein the locking mechanism can be transferred from a first position in which the inner tube 11 and the outer tube 12 of the telescopic rod 10 are held immovably in relation to one another, into a second position in which the inner tube 11 and the outer tube 12 of the telescopic rod 10 are movable in relation to one another in the longitudinal direction of the tubes 11 and 12. There is a certain play between the inner tube 11 and the outer tube 12, such that in a certain region the two tubes 11 and 12 can be rotated in relation to one another around their longitudinal axes.

When the telescopic rod 10 is placed on the operating table O, the telescopic rod 10 can be flexibly supported on the operating table O. The actuating mechanism 15 is disposed on the inner tube 11 directly below the jointed connection 30 with the retractor rod 20. The retractor mounting 21 in this exemplary embodiment is adapted to grip a retractor R in a positive fit. For this purpose, the retractor mounting 21 has an essentially U-shaped recess 22 (FIGS. 8 and 9) into which a frame of a retractor R can be introduced. FIG. 11 shows that the retractor R rests against the inner wall 23 of the recess 22 on three sides and has an elastically pre-stressed tongue 24 [which has] a locking projection 25 at its distal end, which projection holds the retractor R in a positive fit in the U-shaped recess. The tongue 24 is rotatably mounted on one side of the recess 22, and is pre-stressed by a compression spring 26 such that its distal end with the locking projection 25 is pressed toward the recess 22. Further, the tongue has an actuating projection 28 at its proximal end 27, which projection can be actuated (pressed) by the surgeon to release the retractor R from the retractor mounting.

In this exemplary embodiment, the jointed connection 30 between the inner tube 11 of the telescopic rod 10 and the retractor rod 20 also forms a hinge joint. Here the actuating mechanism 15 is a ring which is movable relative to the telescopic rod 10 in the direction of the hinge joint 30 in order to convert the latching mechanism from the first position to the second position.

The telescopic retractor holder according to this exemplary embodiment is employed as follows. After the surgeon has opened the sternum of the patient P and has inserted and spread the retractor R, he grips the telescopic retractor mounting in the region of the hinge joint 30 and sets the telescopic rod 10 on the operating table O. He then applies his thumb to the hinge joint 30 and grips the ring 15 with his index finger and ring finger and pulls said ring toward his thumb so as to release the lock on the telescopic rod 10. He then adjusts the length of the telescopic rod 10 to the requirements of the patient P, which means essentially to the size of the thorax of the patient P. While with one hand he continues to hold the retractor holder in the region of the hinge joint, with the other hand he guides the retractor mounting 21 in the direction of the retractor R (or the fixing location provided on the retractor R). This is ordinarily one of the two legs of a retractor which extend parallel to the sternum incision and hold blades. He then inserts the retractor R into the retractor mounting 21 by pushing the retractor mounting 21 over the retractor R. The surgeon can now raise the side of the sternum of the patient P on the side on which the retractor holder is positioned by again actuating the actuating mechanism and raising the jointed connection 30. Because the retractor rod is fixedly (albeit releasably) connected to the retractor R, and thus forms a kind of cantilever arm of the retractor R, one has an advantageous lever for raising or exposing one half of the sternum. In this way, the surgeon needs to exert less force to expose the sternum. Further, there is the relevant fact that the ribs can be more easily bent outward (with substantially less force) than they can be bent into the thoracic cavity. Thus, when the retractor rod is raised, the ribs on the side of the retractor to which the telescopic retractor holder is applied are bent outward. This creates a force which tends to press the ribs on the other side of the thorax inward. The ribs have substantial resistance to such deformation, so that to some extent they serve as counter-members for the exposure of the opposing halves of the sternum.

If the surgeon now releases the retractor holder, the retractor holder is supported against the operating table O and holds up the said half of the sternum. The above-described rotations α and β which occur due to misalignment of the patient and the different characteristics of the caudal and cranial ribs may be equalized by a rotation and a slight tipping of the retractor mounting.

In the course of the surgery, if the surgeon wishes to raise the other side of the patient's P sternum, he first brings the retractor R into an nearly horizontal position by appropriately reducing the length of the telescopic rod, releases the retractor mounting from the retractor R, places the retractor holder on the other side of the patient P, attaches the retractor mounting to the other leg of the retractor, and again lengthens the telescopic rod. The surgeon can do all this without leaving the sterile area. A skilled surgeon needs only one hand for the entire procedure, and never needs to use his other hand to attach the retractor mounting to the retractor R or to release the retractor R from the retractor mounting.

A second exemplary embodiment of the invention is described below with reference to FIG. 2. The second exemplary embodiment differs from the first exemplary embodiment only in the support plate 70 disposed on the free end of the telescopic rod 10. With the first exemplary embodiment, it is possible that the free end of the telescopic rod can slip away when the friction between the telescopic rod 10 and the operating table O is insufficient, or when someone bumps against the retractor holder. In order to improve the support of the retractor holder on the operating table O, a support plate 70 is provided which preferably has the shape of a spherical surface on its under side. This makes it less likely that the retractor holder will slip away.

A third exemplary embodiment of the invention is described below with reference to FIG. 3. The third exemplary embodiment differs from the second only in that, in the second exemplary embodiment, the inner tube 11 is the upper tube and the outer tube 12 is the lower tube, whereas in the third exemplary embodiment, the inner tube 11 is the lower tube and the outer tube 12 is the upper tube. An alternative (not shown) to the support plate 70 of the second and third exemplary embodiments is a similar plate which rests against the operating table O with its flat side and is attached to the free end of the telescopic rod via a ball joint.

A fourth exemplary embodiment of the invention will be described below with reference to FIG. 4. The third exemplary embodiment differs from the third second by an attaching mount 40 which is laterally connected to the outer tube 12 of the telescopic rod 10. The attaching mount 40 is connected to the telescopic rod 10 via a hinge joint 45, and has a U-shaped member which can fit over a rail on the operating table. The manner in which the mount 40 fits over the rail is that it rests against the operating table rail on three sides, but is slidable along said rail. Also, the telescoping rod is longer than those in the preceding exemplary embodiments, so that it extends beyond the operating table rail toward the floor. Otherwise, the structure and functioning of this exemplary embodiment corresponds to those of the first through third exemplary embodiments; the retractor holder can be tilted toward the patient P via the hinge joint 45, and tilting along the longitudinal axis of the operating table (angle β) is also possible by slightly rotating the telescopic rod with respect to the operating table rail, and a misalignment of the retractor R in the horizontal direction (angle α) can be equalized by simultaneously shifting and tilting the fastening mounting. The one-handed transfer from one side of the patient P to the other side proceeds analogously to that of the preceding exemplary embodiments.

A fifth exemplary embodiment of the invention is described below with reference to FIG. 5. This particularly advantageous exemplary embodiment differs from the fourth exemplary embodiment in that the fastening mounting is not flexible but is rigidly attached to the telescopic rod 10. Consequently, the telescopic rod 10 cannot be tilted toward the patient P. With one of the preceding exemplary embodiments, when the surgeon raises a half of the sternum by increasing the length of the telescopic rod 10, the rod will tilt toward the patient P (provided that the patient is not moved), because the length is fixed between the sternum and the jointed connection 30, between the retractor rod 20 and the telescopic rod 10. In this instance, the sternum is positioned midway between the two spread-open sternum halves, thus approximately the geometric midpoint of the retractor. If, in accordance with this exemplary embodiment, the telescopic rod 10 cannot tilt toward the patient P when the rod 10 is lengthened, the patient will be pulled toward the telescopic rod 10, which will result in appreciable forces on the patient P and may thus cause traumatic injuries. Accordingly, with this exemplary embodiment, the retractor rod 20 is designed to be freely telescopic, so that no lateral forces are exerted on the patient P. For this purpose, the inner part 20B of the retractor rod 20 can be moved back and forth over a sufficiently large range in the outer part 20A of the retractor rod 20. A locking device on the telescopic retractor rod 20 is not necessary, but may be advantageous for the purpose of secure and space-saving storage. With the described telescopic retractor rod 20B, if the inner rod is rotatable with respect to the outer rod 20A, rotation through angle β on this path can be absorbed.

A sixth exemplary embodiment of the invention is described below with reference to FIG. 6 and FIGS. 8 to 13.

This sixth exemplary embodiment exhibits a telescopic retractor holder with a telescopic rod 10 having an inner tube 11, an outer tube 12, and a locking mechanism, along with a retractor rod 20 having a retractor mounting 21 on one end. The retractor mounting 21 is designed to grip a retractor R releasably and with positive fit and is identical to those of the preceding exemplary embodiments. On its other end, the retractor rod 20, having a fixed length, i.e. being non-telescopic, is attached to the inner tube 11 of the telescopic rod 10 via a ball joint 30. An actuating ring 15 by means of which the locking mechanism is changed from a locked state to a released state is attached to the inner tube 11 of the telescopic rod 10 immediately below the ball joint 30.

The telescopic retractor holder according to the sixth exemplary embodiment has an attaching mechanism 50 which has an attaching rail 51 and is designed to be releasably attached to a rail 100 of an operating table O. In the present case, the attaching mechanism 50 is mounted laterally on the operating table rail and is held in place there with the aid of a screw. The outer tube 12 of the telescopic rod 10 has a fastening mounting 60 which can slide longitudinally on the attaching rail 51. The attaching rail 51 has a cross section which is a combination of an essentially circular surface 52 and a second surface 53 which is a segment of a circle such that the two surfaces together form a shape that is essentially a keyhole. The fastening mounting 60 of the telescopic rod 10 has a recess 61 with an inner wall 62 and two side walls 63, with the cross section of the recess 61 essentially corresponding to the cross section of the attaching rail 51, thus also essentially having the shape of a keyhole. Additionally, the inner wall 62 of the recess 61 when pushed over the attaching rail 51 has at least one undercut structure. In this manner, the tractive forces present in the telescopic rod 10 can be transmitted from the fastening mounting 60 to the attaching rail 51 of the attaching mechanism 50. The lateral walls 63 of the recess 61 are spaced from and directed toward the lateral surfaces 54 of the second surface 53 of the cross sectional surface of the attaching rail 51 such that rotation of the telescopic rod 10 relative to the attaching rail 51 is limited to a predetermined angular range, as is apparent in particular from FIGS. 10 and 11. The angles and distances of the lateral surfaces 54 of the attaching rail 51 with respect to the lateral walls 63 of the fastening mounting 60, as shown in FIGS. 10 and 11, prevent the retractor holder from tilting away, and also prevent the retractor holder from falling onto the patient P. This means that when the telescopic retractor holder is pushed onto the attaching rail 51 of the attaching mechanism 50, said retractor holder can rotate in only a predetermined angular range with respect to the attaching rail 51. Thus, it cannot injure the patient P by tipping or falling, and it cannot tip or fall outside the sterile area into an unsterile area (or, at least, an area that is not reliably sterile). When, in addition, the retractor mounting 21 is attached to the retractor R, it is also not possible for the telescopic rod 10 to accidentally slip away.

With regard to this exemplary embodiment, the functioning of the invention will again be described in detail with reference to FIGS. 11 to 13. With this exemplary embodiment, two different procedures are possible.

The first procedure is very similar to those already described. The telescopic retractor holder has two attaching mechanisms 50, each of which is mounted on the operating table rails 100 on one side of the operating table before the surgery begins.

First, the surgeon opens the sternum of the patient P, applies the retractor R, and spreads the sternum of the patient P with the retractor R. In order to expose one side of the sternum, that is to raise said side, to free and prepare an artery running along the inner wall of the thorax, the surgeon applies the telescopic retractor holder to the attaching rail 51 on one side of the half of the sternum which is to be raised. He then attaches the retractor R to the retractor holder with the aid of the retractor mounting 21. If the surgeon now wishes to raise the side of the sternum on which the retractor holder has been placed, he lengthens the telescopic rod 10 using the single-hand actuating mechanism 15, as a result of which the retractor rod 20 causes the retractor R to incline. In the process, the distal leg of the retractor R is supported against the half of the sternum which is not to be raised, and the proximal leg of the retractor R pulls upward on the half of the sternum which is to be raised.

After a successful procedure on this side of the patient P, the telescopic rod 10 is shortened until, as much as possible, there is no moment on the retractor mounting 21. In this state, the retractor R can be particularly easily released from the retractor R with the use of only one hand. The surgeon thus releases the retractor R from the retractor mounting 21 and slides or pulls the telescopic retractor holder laterally in the longitudinal direction of the attaching rail 51 of the attaching mechanism 50. Then, without having to grip around the telescopic retractor holder, he slides said holder on the fastening rail 51 of the second fastening mechanism 50 which has been mounted opposite to the patient P, and applies the retractor mounting 21 to the retractor R, namely the opposite leg of the retractor R that is opposite to that previously used, and he can now raise the other half of the sternum by lengthening the telescopic rod 10. In this way, without the assistance of another person, the surgeon can expose and raise the desired halves of the sternum in succession, without engendering the risk of unsterility in the process. With this first procedure, the surgeon has the advantage that the telescopic retractor holder is always positioned on the side of the patient P on which the sternum is currently being raised. Understandably, the surgeon is more likely to be on the side of the sternum half which is not raised, because this gives him the best view of the region of the thorax of the patient P to be prepared.

According to the second procedure, the surgeon needs only a single attaching mechanism 50, which is attached to an operating table rail 100 on one side of the operating table O before surgery begins. First, the surgeon proceeds as with the first procedure: he attaches the telescoping retractor holder to the attaching mechanism 50, attaches the retractor R to the retractor mounting 21, and lengthens the telescoping rod 10 to raise the half of the sternum corresponding to the side on which the retractor holder is positioned. If the surgeon now desires to raise the other side of the sternum, it is unnecessary for him to unmount the telescopic retractor holder and re-mount it on the other side of the patient P; instead, he only needs to simply shorten the telescopic rod 10 above the retractor mounting 21 which is momentarily essentially free. This will cause pressure to be applied to the side of the sternum on which the retractor holder is disposed. Since, as described above, it is more difficult to press ribs inward than to pull them outward, the half of the sternum to which the pressure is applied remains essentially in its initial position (spread but not rotated or raised or lowered), and the other half of the sternum becomes raised and exposed. In this case, the telescopic retractor holder is indeed disposed on the side on which the surgeon stands, but because the telescoping rod 10 has been substantially shortened it does not interfere with the surgeon in the course of the surgery. In a situation where a half of the sternum is exposed by shortening the telescoping retractor holder, the forces exerted on the patient P are admittedly slightly greater than when the exposure is caused by lengthening the telescopic retractor holder, but this factor is not significant for most patients. With this second procedure, the surgeon does not need to re-install the telescoping retractor holder, and therefore saves the time for this re-installation and saves a second attaching mechanism 50.

A seventh exemplary embodiment of the invention is described below with reference to FIG. 7. The seventh exemplary embodiment differs from the sixth in that the fastening mounting 60 is not positioned on the free end of the telescopic rod 10 but is mounted laterally on the telescoping rod 10, namely on the outer tube 12. In addition, a hinge joint rather than a ball joint is provided between the retractor rod 20 and the telescopic rod 10.

Additional advantages of the invention and individual exemplary embodiments of the invention are described below.

The use of one or more attaching mechanisms 50 has the advantage that the surgeon does not risk damaging other surgical items with the attaching rail 51. If the surgeon has the telescoping retractor holder mounted directly on the covered operating table rail, he cannot know whether the supporting parts of the retractor holder are free from cables, tubing or light cables, which might be hidden under the cloths covering the operating table O, and which the surgeon may disconnect.

In FIG. 10, the geometry of a fastening mounting 60 and an attaching rail 51 are shown. Here the projections which are formed on the transitions from the inner wall 62 to the lateral walls 63, 63 form undercut structures which engage the transition of the outside surface 55 to the lateral surfaces 54, 54 of the attaching rail 51. As seen from FIG. 10, the lateral surfaces 54, 54 are mutually parallel, whereas the lateral walls 63, 63 subtend a certain angle between them. In this way, a certain freedom of movement of the telescopic rod 10 is provided around the attaching rail 51, which allows adjustment to the varying geometry of the telescoping retractor holder during use. At the same time, the lateral walls 63, 63 delimit a rotation of the telescoping rod 10 around the attaching rail 51, so that the upper region of the telescoping rod 10 cannot tip into the non-sterile area and also cannot tip onto the patient. It is not necessary that the angle between the two side walls 63, 63 be symmetrical with the longitudinal axis of the telescoping rod 10; rather, it may be adjusted to meet requirements.

To facilitate lateral sliding of the fastening mounting 60 on the attaching rail 51, the attaching rail may be narrowed at one or both ends. This facilitates sliding, particularly in single-hand operation. In addition, the surgeon will not be tempted to grip the telescoping rod 10 in the region of the fastening mounting 60, that is, in a non-sterile region.

It is not necessary that the fastening mounting be open at the free end of the telescoping rod 10, but rather it may be oriented at any desired angle with respect to the longitudinal axis of the telescopic rod 10. For example, the fastening mounting 60 may open transversely to the longitudinal axis of the telescopic rod 10 in the direction of the operating table O, and the fastening rail may be directed away from the operating table O, in particular rotated by 90°.

In addition, in place of a two-piece telescoping mechanism, a three-piece or multiple-piece telescoping mechanism may be employed in the telescoping retractor holder. The inner tube 11 may be non-rotatable with respect to the outer tube 12, or it may be rotatable only within a certain range.

Additional combinations of the individual features are possible and numerous additional modifications and refinements will be apparent to one skilled in the art, from a reading of the present specification and the following claims and drawings.

1. A telescoping retractor holder, with a telescoping rod having an inner tube, an outer tube and a locking mechanism, and
a retractor rod which
has a retractor mounting at one of its ends designed to releasably engage a retractor, and at its other end is attached to the telescopic rod via a jointed connection,
wherein the telescopic rod
can be supported on an operating table and
has an actuating mechanism by means of which the locking mechanism may be changed from a first position in which the inner tube and outer tube of the telescopic rod are held immovably with respect to each other, into a second position in which the inner tube and outer tube of the telescopic rod are movable with respect to each other in the longitudinal direction of the tubes.

2. The telescopic retractor holder according to claim 1, wherein
the telescoping rod can be flexibly mounted on an operating table.

3. The telescopic retractor holder according to claim 1, wherein the retractor rod is freely telescopic.

4. The telescopic retractor holder according to claim 1, wherein the actuating mechanism is positioned in the region of the jointed connection with the retractor rod.

5. The telescopic retractor holder according to claim 1, wherein
the inner tube of the telescoping rod can be supported on an operating table;
the outer tube of the telescoping rod is connected on one end to the retractor rod via the jointed connection; and
the actuating mechanism is arranged in the region of the other end of the outer tube.

6. The telescopic retractor holder according to claim 1, wherein the retractor mounting is designed to engage a retractor with positive fit.

7. The telescopic retractor holder according to claim 6, wherein
the retractor mounting has an essentially U-shaped recess into which a frame of a retractor can be inserted, so that the retractor frame is seated at least partially against the inner wall of the recess on at least two sides, and has an elastically pre-stressed tongue, wherein the tongue has on its distal end a locking projection which is designed to rest against a retractor frame when it is inserted, thereby producing a positive fit engagement with the retractor frame.

8. The telescopic retractor holder according to claim 7, wherein
the tongue is rotatably mounted on one side of the recess and is pre-stressed by an elastic component such that its distal end with a locking projection is pressed toward the recess.

9. The telescopic retractor holder according to claim 8, wherein
the elastic component is a compression spring which presses against the proximal end of the tongue.

10. The telescopic retractor holder according to claim 7, wherein
the tongue has an actuating projection at its proximal end for releasing a retractor from the retractor mounting.

11. The telescopic retractor holder according to claim 1, wherein
the jointed connection which connects the retractor rod to the telescopic rod is a hinge joint.

12. The telescopic retractor holder according to claim 1, wherein
the jointed connection which connects the retractor rod to the telescopic rod is a ball joint.

13. The telescopic retractor holder according to claim 1, wherein the actuating mechanism is a ring which is movable relative to the telescopic rod to change the locking mechanism from the first position to the second position.

14. The telescopic retractor holder according to claim 1, wherein
the telescopic rod has an essentially U-shaped attaching mount to be mounted on an operating table rail by applying it from above.

15. The telescopic retractor holder according to claim 14, wherein
the cross section of the attaching mount is adjusted to a cross section of an operating table rail such that in the mounted state the attaching mount rests at least partially against both walls of the operating table rail, and wherein
the attaching mount is connected to the telescopic rod via a jointed connection.

16. The telescopic retractor holder according to claim 15, wherein
the jointed connection between the telescopic rod and the attaching mount is configured such that a rotational movement is possible between the telescopic rod and the attaching mount only within a predetermined limited angular range.

17. The telescopic retractor holder according to claim 1, wherein
the telescoping retractor holder has at least one attaching mechanism which has an attaching rail and is designed to be releasably attachable to an operating table rail, wherein
the telescoping rod has a fastening mounting which can be mounted on said attaching rail and is slidable thereon.

18. The telescopic retractor holder according to claim 17, wherein
the attaching rail has a cross section which consists of an essentially circular surface and another surface,
the fastening mounting of the telescopic rod has a recess having an inner wall and two side walls, wherein the cross section of the recess substantially corresponds to the cross section of the attaching rail;
with the attaching rail, the inner wall of the recess in the applied state forms at least one undercut structure which is able to transfer tractive forces from the telescopic rod to the attaching rail; and
the side walls of the recess are spaced from and oriented with respect to the lateral surfaces of the other surface of the cross sectional surface of the attaching rail such that rotation of the telescopic rod relative to the attaching rail is limited to a predetermined angular range.

19. The telescopic retractor holder according to claim 14, wherein
the attaching mounting is arranged at one end of the telescoping rod.

20. The telescopic retractor holder according to claim 14, wherein
the retractor rod is attached to the inner tube of the telescopic rod;
the actuating mechanism is provided at the inner tube of the telescoping rod; and
the attaching mounting is provided at a side wall of the outer tube of the telescoping rod.

21. The telescopic retractor holder according to claim 1, wherein
the telescoping rod has at least one additional middle tube, wherein the at least one additional middle tube positioned between the inner tube) and the outer tube and together with this forms a multiply-telescoping rod.
